(19) United States
(12) Patent Application Publication
(10) Pub. No.: US 2011/0184251 A1
(43) Pub. Date: Jul. 28, 2011

(54) BONE MINERAL DENSITY RATIOS AS A PREDICTOR OF OSTEOARTHRITIS

(75) Inventors: Grace H. Lo, Houston, TX (US); Timothy E. McAlindon, Belmont, MA (US)

(73) Assignee: TUFTS MEDICAL CENTER, MEDFORD, MA (US)

(21) Appl. No.: 13/057,892

(22) PCT Filed: Aug. 25, 2009
(86) PCT No.: PCT/US09/54882

§ 371 Date: Apr. 1, 2011

Related U.S. Application Data
(60) Provisional application No. 61/092,246, filed on Aug. 27, 2008.

(51) Int. Cl. A61B 5/00; G01N 23/06

(52) U.S. Cl. 600/300; 378/54

(57) ABSTRACT

The present invention relates to systems, compositions, and methods for using bone mineral density ratios as a predictor of osteoarthritis. In particular, the present invention relates to comparing ratios of bone mineral density involving bones that are periarticular to determine a risk assessment for features of osteoarthritis.

### CROSS REFERENCE TO RELATED APPLICATIONS

This application claims priority to provisional patent application 61/092,246, filed Aug. 27, 2008, which is herein incorporated by reference in its entirety.

### GOVERNMENT SUPPORT

This invention was made with government support under grant number R01 AR051361-01A1 awarded by the National Institutes of Health. The government has certain rights in the invention.

### FIELD OF THE INVENTION

The present invention relates to systems, compositions, and methods for using bone mineral density ratios as a predictor of osteoarthritis. In particular, the present invention relates to comparing ratios of bone mineral density involving bones that are periarticular to determine a risk assessment for features of osteoarthritis.

### BACKGROUND OF THE INVENTION

Osteoarthritis (OA, also known as degenerative arthritis, degenerative joint disease), is the most common form of arthritis, affecting at least 10% of the population over the age of 65, and at present there is little available in the treatment of this condition, notwithstanding NSAIDs and total joint replacements. Disability from OA is one of the leading causes of disability in the elderly. Unfortunately, the pathophysiology of this disease has not been clarified to date.

The diagnosis of osteoarthritis (OA) is primarily based on history and physical examination. Usually, the clinical features that a patient exhibits—specifically the symptoms he complains of and the signs noted on examination—are sufficient to make the diagnosis of OA. To date, the most common means of confirming a diagnosis of OA is by obtaining plain radiographs of the affected joint; however, it is well-established that radiographs are notoriously insensitive to the detection of OA. Particularly because few effective treatments are available to treat this condition, identification of a measure that could predict the development of OA would be very useful. Additional methods are needed to assess early signs of osteoarthritis and to identify those who are at high risk of developing OA.

### SUMMARY OF THE INVENTION

The present invention relates to systems, compositions, and methods for using bone mineral density ratios as a predictor of osteoarthritis. In particular, the present invention relates to comparing ratios of bone mineral density involving bones that are periarticular to determine a risk assessment for features of osteoarthritis.

Embodiments of the present invention provide inexpensive, non-invasive systems and methods for screening, diagnosing and monitoring the progression of osteoarthritis. For example, some embodiments of the present invention provide research and clinical systems and methods for utilizing BMD ratios for identifying subjects at risk of developing osteoarthritis and having progressive osteoarthritis. In some embodiments, the present invention provides systems and methods for screening compounds useful in the treatment or prevention of osteoarthritis.

Accordingly, in some embodiments, the present invention provides a method of determining a risk of osteoarthritis in a subject, comprising: determining one or more ratios of bone mineral density in a region of a joint bone (e.g., knee bone) of the subject selected from, for example, medial bone mineral density: lateral bone mineral density, medial proximal bone mineral density: medial bone mineral density, or proximal medial bone mineral density: distal medial bone mineral density; and identifying subjects at risk of developing osteoarthritis when the ratio of bone mineral density is increased relative to the level in control subjects (e.g., subjects that do not have osteoarthritis, data from the same subject at an earlier time period, etc.). In some embodiments, the bone mineral density is determined using dual X-ray absorptiometry (DXA). In some embodiments, a medial proximal bone mineral density: medial bone mineral density greater than 1.32 is indicative of subjects at risk of developing osteoarthritis.

In further embodiments, the present invention provides a method of monitoring progression of osteoarthritis in a subject diagnosed with osteoarthritis, comprising: determining one or more ratios of bone mineral density at an initial time point in a region of a joint bone (e.g., knee bone) of the subject selected from, for example, medial bone mineral density: lateral bone mineral density, medial proximal bone mineral density: medial bone mineral density, or proximal medial bone mineral density: distal medial bone mineral density; determining a second one or more initial ratios of bone mineral density at a later time point in a region of a joint bone of the subject selected from, for example, medial bone mineral density: lateral bone mineral density, medial proximal bone mineral density: medial bone mineral density, or proximal medial bone mineral density: distal medial bone mineral density; and identifying subjects as having a progression of osteoarthritis when the ratio of bone mineral density is increased at the later time point relative to the initial time point. In some embodiments, a medial proximal bone mineral density: medial bone mineral density greater than 1.32 is indicative of subjects at risk of having progression of osteoarthritis. In some embodiments, the bone mineral density is determined using dual X-ray absorptiometry (DXA). In some embodiments, the later time point is approximately one year after the initial time point. In some embodiments, the method further comprises the step of determining a further one or more initial ratios of bone mineral density at later time points in a region of a joint bone of the subject selected from, for example, medial bone mineral density: lateral bone mineral density, medial proximal bone mineral density: medial bone mineral density, or proximal medial bone mineral density: distal medial bone mineral density. In some embodiments, the later time points are spaced approximately one year apart. In some embodiments, the method further comprises the step of administering a test compound or other intervention to the subject.

Additional embodiments of the present invention provide a system, comprising: an imaging device; and computer hardware and software configured to calculate bone mineral density in a region of a joint bone of a subject selected from, for example, medial bone mineral density: lateral bone mineral density, medial proximal bone mineral density: medial bone mineral density, or proximal medial bone mineral density: distal medial bone mineral density; and a user interface configured to display the bone mineral density ratios. In some embodiments, the imaging device is a DXA device. In some embodiments, the imaging device determines the region of a joint bone. In some embodiments, the computer hardware maintains a database of bone mineral density ratio. In some embodiments, the bone mineral density ratios in the database are tagged with subject identification tags and time stamp tags.

Additional embodiments are described herein.

### DESCRIPTION OF THE FIGURES

FIG. 1 shows a DXA image of the knee.

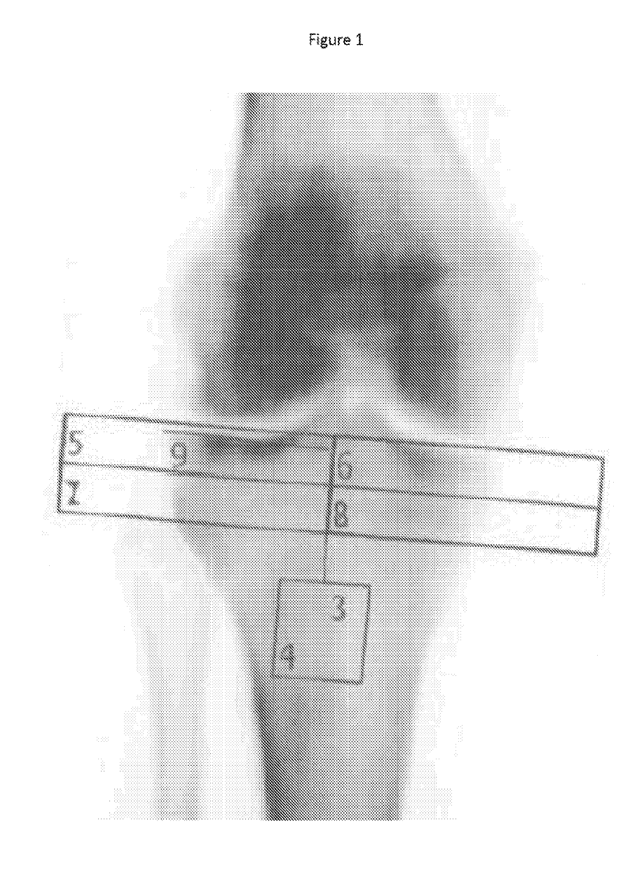

FIG. 2 shows a DXA image of the knee with labels identifying the medial and lateral zones (both proximal, distal and total).

FIG. 3 shows a trabecular MRR sequence.

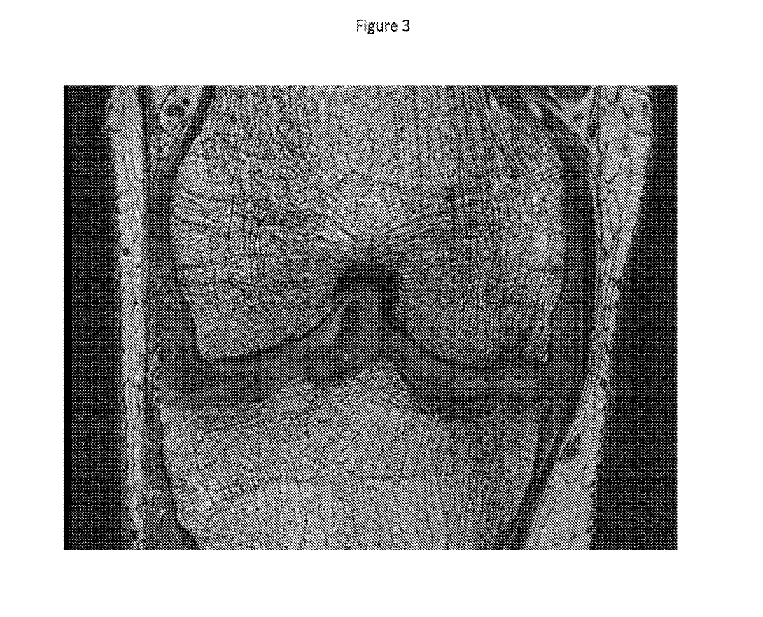

FIG. 4 shows bone volume fraction vs. bone mineral density (BMD).

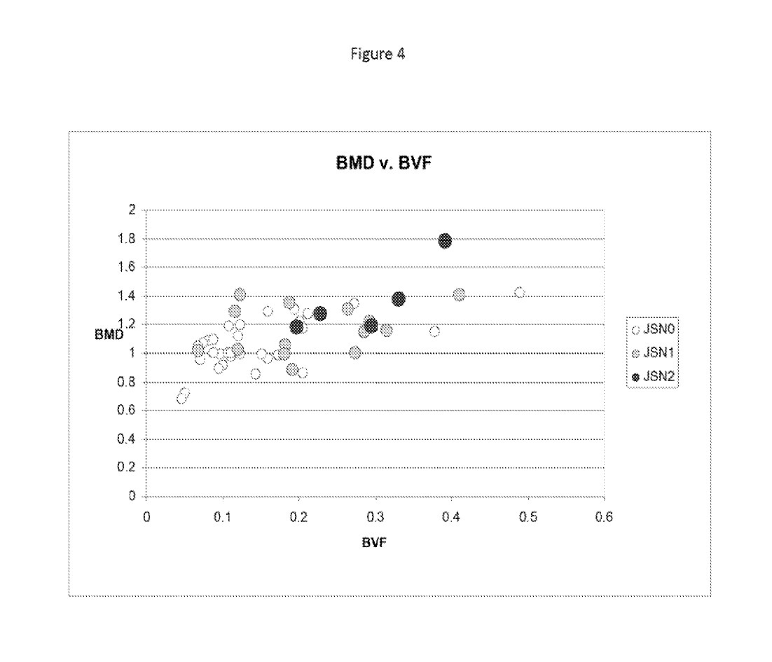

### DEFINITIONS

As used herein, the term “substantially” refers to greater than 75% (e.g., greater than 80%, 85%, 90%, 95%, 98%, or 99%).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having osteoarthritis in a joint” refers to a subject that presents one or more symptoms or risk factors indicative of osteoarthritis (e.g., pain on walking, family history, etc.) or is being screened for osteoarthritis (e.g., during a routine physical).

As used herein, the term “a subject diagnosed with osteoarthritis in a joint” refers to a subject that has been diagnosed with osteoarthritis based on one or more diagnostic assays (e.g., MRI of the joint, x-ray, physical examination, etc.)

### DETAILED DESCRIPTION OF THE INVENTION

The present invention relates to systems, compositions, and methods for using bone mineral density ratios as a predictor of osteoarthritis. In particular, the present invention relates to comparing ratios of bone mineral density involving bones that are periarticular to determine a risk assessment for features of osteoarthritis.

Animal models of OA show that increases in thickness of the subchondral plate occur early in, or even antedate, the development of cartilage loss (Radin et al., J Orthop Res 1984; 2:221-34; Carlson et al., J Orthop Res 1994; 12:331-9). Studies in mice, rabbits and dogs (Benske et al., Acta Orthop Scand 1988; 59:536-41; Newberry et al., J Orthop Res 1997; 15:450-5; Burr D B, J Rheumatol Suppl 2004; 70:77-80) indicate that thickening and remodeling of the subchondral plate is closely linked to cartilage destruction. The observation that lower systemic bone mineral density (BMD) is strongly associated with knee OA progression indicates that lower mineral content is also detrimental (Zhang et al., J Rheumatol 2000; 27:1032-7).

According to Wolff's Law, trabecular size and orientation reflect internal patterns of tensile and compressive stress (Wolff, Clin Orthop Relat Res 1988:2-11). The fact that these forces increase with proximity to the articular surface indicates that the ability of the trabecular network to absorb loads is inversely related to intra-trabecular spacing, size and connectivity. In other words, multiple small trabecular compartments may be better able to attenuate loads than a smaller number of large compartments. Furthermore, it appears that the overall pattern of trabecular orientation contributes to pressure kinetics by influencing the directionality of intra-osseous fluid flow (Nauman et al., Ann Biomed Eng 1999; 27:517-24).

Elevated peri-articular BMD as measured by DXA reflects an increase in the amount of mineralized bone in that region (Pastoureau et al., Osteoarthritis Cartilage 1999; 7:466-73). At a trabecular level this could result from an increase in thickness and volume of the individual trabeculae, and/or spatial compression or collapse of a number of trabeculae into a smaller area. Both are expected to impair the biomechanical properties of the bone. Thus, elevated tibial periarticular BMD indicates a liability for development or progression of knee OA.

The medial:lateral (M:L) tibial BMD ratio has construct validity as an indicator of knee OA. It correlates with knee OA severity (Akamatsu et al., Clin Orthop 1997:207-14; Wada et al., Rheumatology (Oxford) 2001; 40:499-505) and with compartment-specific radiologic features including joint space narrowing (JSN), osteophytes and sclerosis (Lo et al., Osteoarthritis Cartilage 2006; 14:984-90). The M:L BMD ratio is more sensitive because it retains an association with radiographic knee even among knees that do not exhibit radiographic sclerosis (Akamatsu et al., supra). Furthermore, the M:L BMD ratio is associated with subchondral pathologies such as bone marrow lesions, which are themselves associated with OA progression (Akamatsu et al., supra; Lo et al., Arthritis Rheum 2005; 52:2814-21; Carbone et al., Arthritis Rheum 2004; 50:3516-25; Felson et al., Ann Intern Med 2001; 134:541-9; Felson et al., Ann Intern Med 2003; 139:330-6; Hunter et al., Arthritis Rheum 2006; 54:1529-35; Pessis et al., Osteoarthritis Cartilage 2003; 11:361-9; Sowers et al., Osteoarthritis Cartilage 2003; 11:387-93).

Analyses of tibial DXA in various settings demonstrated tibial subchondral BMD to be associated with radiographic joint space loss and malalignment, cartilage damage on MRI (Lo et al., Arthritis Rheum 2006; 56:S125), and with risk for progression of functional decline (Smith et al., Arthritis & Rheumatism 2008; 58:S424).

There is also evidence that tibial subchondral BMD predicts risk for subsequent longitudinal progression of knee OA. The predictivity of a single unadjusted measure (i.e. not the ratio) of medial tibial subchondral BMD for subsequent 1-year loss of joint space width measured among 56 patients with knee OA was found to be strongly correlated with the 1-year change in minimum joint space width (r=−0.43, p=0.02). After adjustment for age, sex, body mass index, and baseline joint space width, BMD of the subchondral bone remained predictive of change in joint space width (β=−4.6, p=0.02).

Conversely, the tibial subchondral BMD appears to be responsive to improvements in mechanical loading (Akamatsu et al., Clin Orthop 1997:207-14; Katsuragawa et al., Int Orthop 1999; 23:164-7), a feature not seen in any other OA measure. One investigation evaluated 23 knees with medial compartment OA following high tibial osteotomy (Akamatsu et al., supra). They reported that the medial:lateral BMD ratio decreased sharply in all 23 knees within 1 year after the procedure. Another studied the effect of a valgus knee brace for medial compartment knee OA. After 3 months the lateral:medial subchondral BMD ratio in the braced knees increased (i.e. improved) from an average of 0.69±0.12 to 0.71±0.13, and in unbraced knees from an average of 0.76±0.10 to 0.77±0.10 (Katsuragawa et al., supra).

In some embodiments, the present invention provides research, screening, diagnostic, and prognostic methods and systems for determining and utilizing BMD ratios.

### I. BMD Ratios

Embodiments of the present invention utilize BMD ratios in research and clinical applications. In some embodiments, the present invention utilizes Dual X-ray Absorptiometry (DXA) or other imaging systems to measure Bone Mineral Density (BMD). DXA is a means of measuring BMD that utilizes technology where two X-ray beams with differing energy levels are aimed at the patient's bones (See e.g., U.S. Pat. Nos. 7,415,146, 6,217,214, 6,029,078, 5,785,041, 5,748,705, 5,687,211; each of which is herein incorporated by reference). When soft tissue absorption is subtracted out, the BMD can be determined from the absorption of each beam by bone. Dual energy X-ray absorptiometry (DXA) is the most widely used and most thoroughly studied bone density measurement technology. Common applications of DXA measurements include assessment of osteoporosis. Systems for performing DXA are commercially available, for example, from GE Medical Systems (Waukesha, Wis.) and Hologic (Bedford, Mass.).

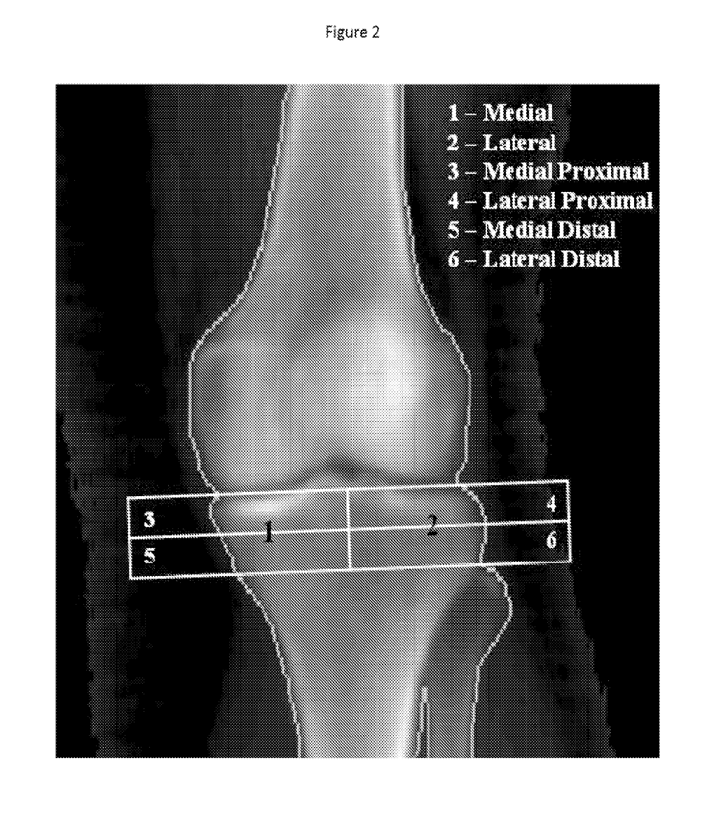

Experiments conducted during the course of development of embodiments of the present invention demonstrated that the strength of relationships of different regions-of-interest (FIG. 2) with OA characteristics varies, indicating that these reflect differing biological processes (Smith et al., Arthritis & Rheumatism 2008; 58:S424).

Embodiments of the present application demonstrate the use of BMD ratios in predicting early joint OA, including early structural changes identified by MRI. Experiments conducted during the development of embodiments of the present invention resulted in the development of ratios of BMD that find use in predicting the risk of developing OA, monitoring the progression of OA, and monitoring the effectiveness of OA treatments (e.g., known and experimental treatments).

In some embodiments, the ratio is the ratio of proximal or closer to the surface bone BMD (e.g., substantially or completely subchondral plate) to distal or deeper bone BMD (e.g., substantially or completely trabecular bone). In other embodiment, the ratio is the ratio of proximal BMD to total BMD. In some embodiments, the ratio of medial to lateral BMD is then calculated (e.g., proximal medial BMD to proximal lateral and distal medial to distal lateral BMD).

In still further embodiments, the BMD ratio is, for example, medial BMD:lateral BMD, medial proximal BMD:medial total BMD, and proximal medial BMD:distal medial BMD.

In some embodiments, one or more (e.g., 2 or more, 3 or more, etc.) ratios may be utilized in combination. In some embodiments, different ratios that are indicative of different risk factors are utilized in combination.

### II. Therapeutic Methods

In some embodiments, the present invention provides methods of screening, diagnosing and monitoring osteoarthritis in a joint. In some embodiments, the present invention provides methods of diagnosing osteoarthritis in a joint. In some embodiments, the present invention provides methods of identifying individuals at risk of developing osteoarthritis in a joint.

The present invention is not limited to a particular cut off for determining the risk of OA. In some embodiments, a threshold of the ratios is indicative of an increased risk of developing OA, although other ratios may also find use. For example, in some embodiments, those with a medial proximal:distal ratio of >1.3 have more pain with walking, difficulty with walking and slower walk time and those with a proximal medial:lateral BMD ratio of >1.4 are associated with medial tibio-femoral articular cartilage damage, with advanced radiographic OA, and with various malalignment (a known risk factor for medial tibiofemoral OA progression).

Accordingly, in some embodiments, the present invention provides methods of comparing ratios of BMD in a joint in order to determine risk of OA. In some embodiments, the regions to be compared are determined by an operator. In other embodiments, determination of the regions is automated (e.g., using software associated with the DXA or other X-ray equipment).

In some embodiments, the ratio is used to identify those at high risk for OA, to monitor progression of OA over time (e.g., measured multiple times per year, once per year, or every 2 or more years). In other embodiments, the ratio is used to monitor therapies over time (e.g., non-steroid anti-inflammatory medication or other OA treatment). In still further embodiments, ratios are used (e.g., in clinical studies) to assess new or candidate OA therapies.

The methods of embodiments of the present invention find use in assessing OA in any number of joints (e.g., knee (e.g., tibial plateau), hip, hand, finger, foot, femur and vertebrae). The methods of embodiments of the present invention are exemplified using the knee. However, the present invention is not intended to be limited to the assessment of OA in the knee.

### III. Systems

In some embodiments, a system is provided comprising imaging devices and appropriate software (e.g., software for data collection, data analysis, imaging device control, user interfaces, etc.). In some embodiments, data analysis software is incorporated into the imaging device (e.g., on a computer processor attached to the imaging device).

In some embodiments, the system provides an image of the joint to be analyzed and the user (e.g., clinician) uses computer software to identify the regions for calculating BMD ratios. In other embodiments, the computer software identifies the regions of interest. In some embodiments, the computer software identifies the regions and the user refines or revises the regions. In some embodiments, the computer software refines the regions of interest over time based on user refinement and adaptive learning algorithms.

In some embodiments, a database of past patient data is used to refine regions of interest and diagnostic and/or prognostic assessments. In some embodiments, the computer software and computer hardware store region of interest information for a specific subject so that identical regions can be compared over time. In some embodiments, the computer software and hardware utilize a registration algorithm to confirm alignment and positioning of the joint of interest in the DXA machine.

In some embodiments, data analysis software provides information in a format that is useful for a clinician without further analysis. For example, in some embodiments, one or more BMD ratio are provided. In some embodiments, a representation (e.g., graphical) of the change in BMD ratios of a given subject over time are provided. In some embodiments, the data analysis software provides a quantitative (e.g., probability) or qualitative assessment of the risk of developing osteoarthritis or the risk of progression of existing osteoarthritis based on the BMD ratios or the change in ratios over time.

### IV. Drug Screening Methods

In some embodiments, the present invention provides methods of screening candidate osteoarthritis compounds. In some embodiments, compounds are administered to a subject diagnosed with osteoarthritis and the progression of disease is monitored over time (e.g., in comparison to a subject not diagnosed with or having symptoms of osteoarthritis).

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

### EXPERIMENTAL

The following examples are provided in order to demonstrate and further illustrate certain preferred embodiments and aspects of the present invention and are not to be construed as limiting the scope thereof.

### Example 1

Overall 50.2% of participants were female, 72.3% were White, 25.5% were Black, 1.1% were Hispanic, and the mean age was 66.1 years. All participants had MRIs, knee DXA scans, and blood samples drawn per protocol. At baseline, there were 14 participants without a trabecular sequence scan or completed MRI. There were 11 (1.8%) participant withdrawals from the ancillary and/or parent study. Measurement of tibial subchondral BMD in the 6 regions of interest was performed on 425 participant knee DXA scans.

This analysis was based on the first 226 enrollees, a sample with mean age 65.3 years (s.d. 9.0), 46.5% were male, 73.5% were White, and mean body mass index (BMI) was 29.9 kg m⁻² (s.d. 5.1). The computation of tibial subchondral BMD and the ratio measures was based on the regions of interest illustrated in FIG. 2. Absolute medial BMD was derived from box 1; the medial:lateral ratio from boxes 1 and 2; and the medial:medial ratio from boxes 3 and 5. The medial:lateral tibial subchondral ratio in the overall group was 1.13 (s.d. 0.15). Knees with greater pain, characterized by WOMAC pain subscale score ≧7 had a mean medial:lateral BMD ratio of 1.17 (s.d. 0.16) compared to the rest of the sample whose mean value was 1.13 (s.d. 0.15), a difference of ˜0.04. Mean medial:lateral BMD ratio values (s.d.) according to presence and severity of radiographic joint space narrowing was as follows: no joint space narrowing 1.08 (0.13); mild/moderate joint space narrowing (grades 1 and 2) 1.17 (0.19); severe joint space narrowing (grades 3) 1.30 (0.14).

There were highly significant relationships of tibial subchondral BMD with structural features—alignment and joint space narrowing (Tables 1 and 2). Absolute medial tibial subchondral BMD is strongly correlated with femoral neck BMD.

TABLE 1
Tibial Subchondral BMD and Knee Alignment

| | Absolute BMD | BMD Ratios | |
|---|---|---|---|
| | β-coefficient; p | β-coefficient; p | |
| | Medial (g/cm2) | Medial:Lateral | Medial:Medial |
| varus | 0.11; p = 0.01 | 0.09; p = 0.003 | 0.02; p = 0.04 |
| normal (ref.) | — | — | — |
| valgus | 0.04; p = 0.3 | −0.04; p = 0.1 | 0.009; p = 0.2 |

TABLE 2
Tibial Subchondral BMD and Joint Space Narrowing

| | Absolute BMD | BMD Ratios | |
|---|---|---|---|
| | β-coefficient; p | β-coefficient; p | |
| JSN Grade | Medial (g/cm2) | Medial:Lateral | Medial:Medial |
| 0 (ref.) | — | — | — |
| 1 or 2 | 0.05; p = 0.1 | 0.09; p < 0.0001 | 0.02; p = 0.002 |
| 3 | 0.21; p < 0.0001 | 0.21; P < 0.0001 | 0.05; p < 0.0001 |

TABLE 3
Tibial Subchondral BMD and Demographics

| | Absolute BMD | BMD Ratios | |
|---|---|---|---|
| | β-coefficient; p | β-coefficient; p | |
| | Medial (g/cm2) | Medial:Lateral | Medial:Medial |
| Age | −0.006; p = 0.0001 | 0.001; p = 0.4 | 0.0007; p = 0.009 |
| Gender | 0.18; p < 0.0001 | 0.05; p = 0.02 | 0.004; p = 0.4 |
| Race | −0.01; p = 0.7 | 0.06; p = 0.01 | 0.006; p = 0.3 |
| BMI | 0.02; p < 0.0001 | 0.002; p = 0.4 | −0.0009; p = 0.05 |
| Femoral BMD | 0.88; p < 0.0001 | 0.05; p = 0.4 | −0.04; p = 0.01 |

There were also relationships with other covariates (Table 3). There is a negative correlation between medial tibial subchondral BMD and age, but that association is in the opposite direction for the medial:medial ratio, possibly on a mechanistic basis. Adjustment of these relationships for structural covariates made little difference to the correlations or level of significance.

It was investigated whether tibial subchondral BMD predicts longitudinal progression in functional ability and pain among individuals with symptomatic knee OA. The sample was drawn from participants in a trial of vitamin D for knee OA had complete function assessments and WOMAC questionnaire reports both at baseline and at their one-year follow-up visit. Walking ability was assessed using a timed 20-meter walk test. Walking pain and difficulty were assessed using the two pertinent WOMAC questions. Worsening on the timed walk test was defined as an increase in walk time between baseline and the one-year assessment and worsening on the WOMAC questions as an increase in reported severity. DXA scans of both knees were obtained at baseline using a GE-Lunar scanner. Subchondral BMD in regions of interest as depicted in FIG. 2 were calculated. Different combinations of tibial subchondral BMD ratio measures including medial:lateral (box 1 versus box 2 in FIG. 2) and within medial compartment (box 3 versus box 1 in FIG. 2) were calculated. Logistic regression with case-based tertiles of BMD ratios as predictors, and worsening of walk time, walking pain, and walking difficulty as outcomes was performed. Analyses were adjusted for age, sex, BMI, and radiographic OA severity (Kellgren-Lawrence grade). These analyses were repeated with the Kellgren-Lawrence grades as a covariate.

The eligible participants (N=89) had a mean age of 64.2 years (+8.7), and mean BMI of 30.3 Kg m⁻² (+5.3) and 67.4% were female. Those with a greater within medial compartment BMD ratio were significantly more likely to report worse pain or exhibit deterioration in walk time at follow-up (Table 4). However, no such relationship was found with the medial to lateral ratio.

The observation that the within medial, but not medial to lateral, tibial subchondral BMD ratio was associated with worsening pain and function indicates that these have differing biological relevance. The influence of the within medial BMD ratio was also independent of radiographic OA severity, indicating that this DXA measure is more predictive of patient outcomes than radiography.

TABLE 4
Within medial compartment tibial subchondral BMD*
and knee OA progression

| | | | Odds | |
|---|---|---|---|---|
| Tertile | Range | | Ratio | (95% CI) |
| | | Worsening of Pain during | | |
| | | Walking | | |
| 1 | 1.03-1.32 | 1/38 (2.6%) | Referent | — |
| 2 | 1.32-1.40 | 6/24 (25.0%) | 12.7 | 1.4-118 |
| 3 | 1.40-1.70 | 10/27 (37.0%) | 26.9 | 3.0-243 |
| | | Worsening of Difficulty | | |
| | | Walking | | |
| 1 | 1.03-1.32 | 2/38 (5.3%) | Referent | — |
| 2 | 1.32-1.40 | 6/24 (25.0%) | 4.9 | 0.8-29 |
| 3 | 1.40-1.70 | 6/27 (22.2%) | 4.9 | 0.8-28.9 |
| | | Worsening of 20-m Walk | | |
| | | Time | | |
| 1 | 1.03-1.32 | 17/38 (44.7%) | Referent | — |
| 2 | 1.32-1.40 | 18/24 (75.0%) | 4.1 | 1.3-13.1 |
| 3 | 1.40-1.70 | 18/27 (66.7%) | 2.7 | 0.9-8.1 |

*Within medial compartment tibial subchondral BMD ratio defined as ROI box 3 vs. box 1 (FIG. 2)

Experiments were conducted to test whether BMD measures of the superficial zone of subchondral bone (0-1 cm), which includes the subchondral plate, have stronger association with radiographic features of knee OA than those of deeper bone (1-2 cm). This study used the same sample of participants as described above. All participants had baseline posterio-anterior semiflexed weight-bearing knee radiographs, knee DXA scans and 1.5 Tesla MRIs taken of their study knee. The knee radiographs were scored for OA severity according to the Kellgren and Lawrence system. Cartilage damage was assessed on the MRIs using the semiquantitative Boston Leeds Osteoarthritis Knee Score (BLOKS), which was developed for this purpose (Hunter et al., Osteoarthritis & Cartilage; 13:241).

Knee DXA scans were used to calculate BMD ratios between medial and lateral, superficial and deep, regions of interest as depicted in FIG. 2. X-rays were scored for Kellgren and Lawrence grade (0-4) and anatomic alignment. To convert the anatomic alignment to mechanical axis (MA), in women 3.5 degrees were subtracted from the anatomic alignment and in men 6.4 degrees, as recommended (Kraus et al., Arthritis Rheum 2005; 52:1730-5). A logistic regression with case-based tertiles of BMD ratios as predictors and moderate-severe cartilage loss in the medial tibiofemoral compartment as the outcome was performed. These tertiles of medial to lateral BMD were also used to predict radiographic OA severity (Kellgren and Lawrence grade 3 or 4) and biomechanical alignment. The analyses was repeated with the superficial medial to lateral BMD (0-1 cm depth) and the deep medial to lateral BMD (1-2 cm depth) as predictors. This analysis found strong associations of the superficial medial to lateral BMD ratio with ipsi-compartmental cartilage damage (by MRI), Kellgren and Lawrence radiographic severity grade, and biomechanical axis (Table 5). Those with the highest superficial medial to lateral BMD ratio all had Kellgren and Lawrence radiographic severity grade 3 or 4 and had varus malalignment. However, the deep medial to lateral BMD ratio showed similar associations with cartilage damage (by MRI), Kellgren and Lawrence radiographic severity grade, and biomechanical axis, albeit less strongly. These indicate that biologically relevant bone changes in knee OA extend beyond the superficial subchondral region.

TABLE 6
Vitamin D Status and Longitudinal Change in Tibial BMD in Knee OA

| | | | | High vs. Low | |
|---|---|---|---|---|---|
| M:L BMD change | | Vitamin D Group | | | Proportional |
| groups | | Low | High | OR (95% CI) | OR (95% CI) |
| Increase | 0.045 to 0.158 | 12/40 (30%) | 8/40 (20%) | 0.6 (0.2-1.6) | 0.4 (0.2-0.9) |
| Stable | ⁻0.038 to 0.041 | 23/40 (57.5%) | 17/40 (42.5%) | Referent | |
| Decrease | ⁻0.177 to ⁻0.03 | 5/40 (12.5%) | 15/40 (37.5%) | 4.2 (1.4-13.1) | |

Using the study sample described above the relationship between vitamin D status and change in knee BMD was analyzed. 25-hydroxyvitamin D levels were obtained at baseline using a commercial HPLC/Mass spectrometry Assay. Knee DXA scans were obtained at baseline and at one year follow-up. These were used to calculate the medial to lateral tibial BMD ratio with a region of interest depth of 2 cm. Those in the highest quartile of change in medial to lateral tibial BMD ratio were defined as increased, those in the middle two quartiles as stable, and those in the lowest quartile as decreased. The median vitamin D level was used to dichotomize those with a high vs. low vitamin D level. To focus on medial compartment disease, knees with moderate-severe cartilage thickness loss in the lateral tibiofemoral compartment were excluded.

Logistic regression analyses were performed using increase in medial to lateral tibial BMD ratio as the outcome and baseline vitamin D as the predictor and repeated using decrease in medial to lateral tibial BMD ratio as the outcome. Table 6 presents the results of these analyses.

TABLE 5
Proximal & Distal Tibial Subchondral BMD Ratios as Predictors of OA Structural Features

| | | Medial tibiofemoral | | | | | |
|---|---|---|---|---|---|---|---|
| | | cartilage damage (MRI) | | X-Ray: KL 3 or 4 | | Varus malalignment | |
| | | Prevalence | OR 95% CI | Prevalence | OR 95% CI | Prevalence | OR 95% CI |
| PROXIMAL | Tertile 1 | 11/68 | — | 20/68 | — | 20/68 | — |
| M:L BMD | Tertile 2 | 14/16 | 36.3 | 13/16 | 10.4 | 14/16 | 16.8 |
| | | | 7.2-182 | | 3.2-29 | | 3.5-81 |
| | Tertile 3 | 12/15 | 20.7 | 15/15 | ∞ | 15/15 | ∞ |
| | | | 5.1-86 | | | | |
| DISTAL | Tertile 1 | 12/59 | — | 20/68 | — | 19/68 | — |
| M:L BMD | Tertile 2 | 13/20 | 7.3 | 13/16 | 9.6 | 15/16 | 6.3 |
| | | | 2.4-22 | | 3.2-29 | | 2.0-20 |
| | Tertile 3 | 12/20 | 5.9 | 15/15 | 28.9 | 15/15 | 6.3 |
| | | | 2.0-18 | | 5.6-148 | | 2.0-20 |

Sufficient change occurred in medial to lateral tibial BMD ratios to detect a difference over one year. Furthermore, baseline vitamin D level predicted change in medial to lateral tibial BMD ratio, such that those with a high level were less likely to have an increase in BMD ratio and more likely to have a decrease (Table 6). Since greater medial to lateral tibial BMD ratios are associated with greater OA severity, this indicates a protective relationship between vitamin D and knee OA.

At baseline, the incidence cohort had 3,284 participants with 6,472 knees available for analysis (96 knees had missing information on radiographic change or symptoms). The breakdown of radiographic changes of OA and frequent knee pain among this sample at baseline was as follows:

| | |
|---|---|
| Normal knees (no radiographic OA and no knee pain) | 2,489 (38%) |
| Radiographic knee OA only (radiographic changes but | 2,870 (44%) |
| no knee pain | |
| Knees pain but no radiographic OA | 785 (12%) |
| Symptomatic knee OA (knee pain plus radiographic | 328 (5%) |
| changes) | |

For the 12-month follow-up exam, symptom data was available for 6153 knees space. Among the knees classified at baseline as “normal”, 12% now have chronic knee pain. Among the knees which at baseline had only radiographic changes, 14% now have chronic pain. This makes them classifiable as having developed incident symptomatic knee OA.

For the 24-month follow-up exam, symptom information was available for the first half of the cohort (3312 knees). Among the subset that had radiographic changes only at baseline, the prevalence of chronic knee pain is approximately 16% (i.e. have symptomatic knee OA).

4Qimaging has developed a fully automated, atlas-based segmentation and analysis system to segment and analyze cartilage and bone features, and other anatomic regions from knee MR image data (Clinical Image Processing and Analysis System—CiPAS). The objective of this analysis was to compare the repeatability and reproducibility of their automated system against an expert radiologist.

The atlas for the automated system was created by manually tracing five subjects' 3D DESS images from the OAI public use dataset and selecting the best performing atlas for further refinement. The repeatability study used 30 randomly selected 3D DESS images from the OAI public use dataset. Of these, 10 were randomly selected to create 40 de-identified images for manual and automated segmentation. The automated segmentations were performed five times with varying initial parameters. The final measurements were obtained by trimming the highest and lowest values and averaging the three remaining measurements.

The reproducibility test used 38 de-identified image sets from 19 subjects who participated in a scan-rescan reproducibility test for the OAI pilot study. These were segmented both semi-manually and automatically. The automated measurements were generated with a trimmed average of five segmentations using varying initial parameters.

Quantitative measurements of the central medial and lateral tibial and femoral cartilage included volume, articulating surface area, subchondral bone surface area, average thickness and standard deviation of average thickness, as well as the bone parameters that we proposed to analyze in this competing revision (see table 7). Those values ranged from 1.7% to 5.37% RMS CV for the automated approach and 3.9 to 7.8% RMS CV for the expert edited approach. The RMS CV includes error from re-positioning and reacquiring the image as well as measurement error. This data set contained a mix of healthy and significantly arthritic subjects and the largest source of variation was from the abnormal subjects.

TABLE 7
Reproducibility (root mean square coefficient of variation) of CiPAS
measurements proposed for use in this competing revision compared to
manual segmentation by an expert

| Quantitative | | |
|---|---|---|
| Measurements of Bone Shape and Signal Parameters | 4Qi | Expert |
| Medial Tibia Subchondral Bone Surface Area | 2.95% | 5.17% |
| Medial Tibia Subchondral Bone Surface Area | 2.46% | 5.69% |
| Medial Tibia Cartilage-bone Contrast | 9.43% | 17.6% |
| Lateral Tibia Cartilage-bone Contrast | 9.14% | 12.4% |
| Medial Tibia Bone Curvature | 31.46% | 24.4% |
| Lateral Tibia Bone Curvature | 46.5% | 55.4% |

The automated atlas based MR image analysis system used in this study to segment the knee into bones and cartilage, and divide the joint in regions and in sub-segments provided repeatable and highly reproducible signal intensity measurements in the medial and lateral weight bearing regions of the knee. These automated tools provide a realistic opportunity to characterize the behavior of structural and compositional changes in cartilage and non-cartilage tissues in OA by analyzing larger populations such as the OAI or other longitudinal datasets.

### Example 2

### Association of Absolute and Relative Tibial Subchondral BMD Measures with Individual Characteristics and Structural Features of Knee Osteoarthritis

The absence of a biomarker for detection and monitoring of knee osteoarthritis (OA) is a fundamental obstacle to the development of structure-modifying interventions. This Example describes the use of tibial subchondral dual x-ray absorptiometry (tsDXA) finds use to generate reproducible measures of knee bone mineral density (BMD). DXA involves low radiation, is easy to opemte, relatively inexpensive and widely available.

This was a cross-sectional analysis of right knee of 226 enrollees into the Osteoarthritis Initiative (OAI) Bone Ancillary Study, who received standardized semiflexed knee radiography and tsDXA. Medial JSN (0-2) and osteophytosis (0-1) was scored on parent study (OAI) baseline images. A goniometer was used to evaluate static alignment on OAI baseline physical exam. Normal alignment was 0 degrees, valgus was <0 and varus was >0. Knee and femoral neck DXAs were obtained at either the OAI 30 or 36 month follow-up visit. tsBMD was computed from the tibial subchondral bone: absolute medial tibial BMD; medial tibial: lateral tibial ratio; medial proximal tibial:medial tibial ratio.

The mean age was 65.3 years (s.d. 9.0), 46.5% were male, 73.5% White, mean BMI was 29.9 kg m⁻² (s.d. 5.1); 25.7% had varus deformity, 38.9% had radiographic medial tibiofemoral JSN of grade 1 or 2 and 84.5% had osteophytosis. The mean (s.d.) values for the tsBMD measures in the sample were: absolute medial 1.16 (0.21); medial:lateral ratio 1.13 (s.d. 0.15); medial:medial ratio 1.14 (s.d. 0.04). The associations of the tsBMD measures with structural features of OA and participant characteristics are presented in the Table 8.

All tsBMD measures were positively associated with the highest grade of medial JSN, a hallmark of knee OA. Further. all tsBMD measures were also associated with varus alignment. Higher absolute medial BMD was associated with younger age, male sex, greater BMI, and systemic BMD. Higher medial:lateral BMD ratio was associated with male sex and white race. Higher medial:medial BMD ratio was associated with older age, lower BMI, and lower systemic BMD.

Each measure of tsBMD is associated with medial JSN and with varus malalignment, indicating that these are meaningful measures of knee OA. However, each is also associated with a different established risk factor of knee OA in the expected direction, absolute medial BMD with BMI, medial:lateral ratio with White race, and medial:medial ratio with age, indicating that each measure might reflect a different process occurring in medial tibiofemoral knee OA. Absolute medial BMD and the medial:medial ratio are associated with systemic BMD in opposite directions.

TABLE 8

| | Absolute BMD | | BMD Ratios | | | |
|---|---|---|---|---|---|---|
| | Medial BMD (g/cm²) | | Medial:Lateral | | Medial:Medial | |
| | Beta | p-value | Beta | p-value | Beta | p-value |
| Medial JSN | — | — | — | — | — | — |
| grade 0 (ref) | | | | | | |
| Medial JSN | 0.05 | 0.1 | 0.09 | <0.0001 | 0.02 | 0.002 |
| grade 1 | | | | | | |
| Medial JSN | 0.21 | <0.0001 | 0.21 | <0.0001 | 0.05 | <0.0001 |
| grade 2 | | | | | | |
| Varus | 0.11 | 0.01 | 0.09 | 0.003 | 0.02 | 0.04 |
| Normal (ref) | — | — | — | — | — | — |
| Valgus | 0.04 | 0.3 | −0.04 | 0.1 | 0.009 | 0.2 |
| Age | −0.006 | <0.0001 | 0.001 | 0.4 | 0.0007 | 0.009 |
| Sex (male) | 0.18 | <0.0001 | 0.05 | 0.02 | 0.004 | 0.4 |
| Race (white) | −0.01 | 0.7 | 0.06 | 0.01 | 0.006 | 0.3 |
| BMI | 0.02 | <0.0001 | 0.002 | 0.4 | −0.0009 | 0.05 |
| Femoral | 0.88 | <0.0001 | 0.05 | 0.4 | −0.04 | 0.01 |
| Neck HMD | | | | | | |

### Example 3

### Increased Medial Tibial Bone Mineral Density (BMD) is Associated with Deterioration in Walking Ability and Pain in Individuals with Knee Osteoarthritis (KOA)

This Example describes a cross-sectional evaluation of baseline data for evaluation of baseline knee BMD data with longitudinal change of functional status. Participants enrolled in an ongoing randomized controlled clinical trial of vitamin D for KOA who had data from both baseline and 1 year follow-up visits and were age 45 and older at time of enrollment and had at least 1 knee with symptomatic radiographic tibio-femoral KOA (K/L grade >2) were eligible for participation. Each participant was assigned a study knee based on K/L grade and pain symptoms Baseline and 1-Year Visits. 20 meter timed walk test (2 trials), timed chair stand test (2 trials of 5 chair stands), WOMAC pain and function questions (Likert) and bilateral knee DXA scans with a GE-Lunar scanner were performed.

Knee BMD has been assessed in multiple ways—one being evaluation of the medial:lateral BMD Ratio (M:L BMD Ratio). Most of the loading within the knee passes through the medial compartment with weight bearing. The preponderance of OA occurs in the medial tibio-femoral compartment.

The following ratios were determined:

### Overall Medial:Lateral BMD Ratio (M:L)

### Proximal M:L BMD Ratio

### Distal M:L BMD Ratio

### Medial BMD Ratios

PM:DM: Ratio of proximal M-BMD to distal M-BMD
PM:TM: Ratio of proximal M-BMD to overall M-BMD

### Cross-Sectional Evaluations

Study knee baseline BMD ratios associations with baseline physical function
Longitudinal evaluations
Study knee baseline BMD ratios associations with change in physical function over one year

### Logistic Regression

### Independent Variable:

Baseline case-based tertiles of:

### Overall M:L BMD Ratio

### Proximal M:L BMD Ratio

### Distal M:L BMD Ratio

### Proximal Medial: Total Medial BMD Ratio (PM:TM)

### Proximal Medial Distal Medial BMD Ratio (PM:DM)

### Cross Sectional Analyses:

Functional Outcomes (Dependent variable)
Walk time (dichotomized at the median)
Chair stand time (dichotomized at the median)

### WOMAC Function

Sum of WOMAC function questions (dichotomized at the median)
Individual WOMAC function question 6 evaluating walking (dichotomized as score of >2)

### Longitudinal Analyses:

Functional outcomes (Dependent variable)
Worsening of walk time (any increase in time)
Worsening of chair stand time (any increase in time)
Worsening total WOMAC function sum (any increase in total score)
Worsening of score on individual WOMAC function question 6 evaluating walking (any increase in individual scores)
P-value for trends
Median BMD Ratio values were used for each case-based tertile group.

### Results: Baseline Characteristics

### N=89

### Mean age: 64.2 (±8.7)

### Mean BMI: 30.3 (±5.3)

67.4% female

### Cross-sectional Analyses

TABLE 9
Medial:Lateral BMD Ratios (M:L)

| | | | Odds Ratio |
|---|---|---|---|
| | | Baseline 20 | |
| | | Meter Walk | |
| | | Time ≧16 | |
| | | seconds | |
| Case-based | Tertile 1 (0.74-1.05) | 14/23 (60.9%) | Referent |
| M:L BMD | Tertile 2 (1.05-1.24) | 15/37 (40.5%) | 0.44 |
| Groups | | | (95% CI 0.15-1.27) |
| | Tertile 3 (1.24-1.72) | 14/29 (48.3%) | 0.60 |
| | | | (95% CI 0.20-1.82) |
| | | | p for trend = 0.47 |
| | | Baseline | |
| | | Chair Stand | |
| | | Time ≧19 | |
| | | seconds | |
| Case-based | Tertile 1 (0.74-1.07) | 16/27 (59.3%) | Referent |
| M:L BMD | Tertile 2 (1.07-1.22) | 16/30 (53.3%) | 0.79 |
| Groups | | | (95% CI 0.27-2.25) |
| | Tertile 3 (1.22-1.72) | 16/32 (50.0%) | 0.69 |
| | | | (95% CI 0.24-1.93) |
| | | | p for trend = 0.48 |

TABLE 10
Medial:Lateral BMD Ratios (M:L)

| | | | Odds Ratio |
|---|---|---|---|
| | | Baseline | |
| | | Total WOMAC | |
| | | function | |
| | | score ≧22 | |
| Case-based | Tertile 1 (0.74-1.07) | 15/28 (53.6%) | Referent |
| M:L BMD | Tertile 2 (1.07-1.26) | 15/36 (41.7%) | 0.62 |
| Groups | | | (95% CI 0.23-1.67) |
| | Tertile 3 (1.26-1.72) | 15/25 (60.0%) | 1.3 |
| | | | (95% CI 0.44-3.87) |
| | | | p for trend = 0.66 |
| | | Baseline | |
| | | Difficulty | |
| | | Walking | |
| | | Score of ≧2 | |
| Case-based | Tertile 1 (0.74-1.07) | 9/28 (32.1%) | Referent |
| M:L BMD | Tertile 2 (1.07-1.29) | 10/38 (26.3%) | 0.51 |
| Groups | | | (95% CI 0.26-2.20) |
| | Tertile 3 (1.29-1.72) | 10/23 (43.5%) | 1.62 |
| | | | (95% CI 0.52-5.10) |
| | | | p for trend = 0.43 |

TABLE 11
Proximal Medial:Distal Medial BMD Ratios (PM:DM)

| | | | Odds Ratio |
|---|---|---|---|
| | | Baseline | |
| | | Walk Time | |
| | | ≧16 seconds | |
| Case-based | Tertile 1 (1.03-1.27) | 14/20 (70.0%) | Referent |
| M-BMD | Tertile 2 (1.27-1.39) | 15/39 (38.5%) | 0.27 |
| Groups | | | (95% CI 0.08-0.85) |
| (PM:DM) | Tertile 3 (1.39-1.70) | 14/30 (46.7%) | 0.38 |
| | | | (95% CI 0.11-1.24) |
| | | | p for trend = 0.17 |
| | | Baseline | |
| | | Chair Stand | |
| | | Time ≧19 | |
| | | seconds | |
| Case-based | Tertile 1 (1.03-1.27) | 16/23 (69.6%) | Referent |
| M-BMD | Tertile 2 (1.27-1.38) | 16/34 (47.1%) | 0.39 |
| Groups | | | (95% CI 0.13-1.19) |
| (PM:DM) | Tertile 3 (1.38-1.70) | 16/32 (50.0%) | 0.44 |
| | | | (95% CI 0.14-1.35) |
| | | | p for trend = 0.19 |

TABLE 12
Medial:Lateral BMD Ratios (M:L)

| | | | Odds Ratio |
|---|---|---|---|
| | | Worsening | |
| | | of 20-meter | |
| | | Walk Time | |
| | | (Objective) | |
| Case-based | Tertile 1 (0.74-1.09) | 17/32 (53.1%) | Referent |
| M:L BMD | Tertile 2 (1.09-1.24) | 18/28 (64.3%) | 1.59 |
| Groups | | | (95% CI 0.56-4.49) |
| | Tertile 3 (1.24-1.72) | 18/29 (62.1%) | 1.44 |
| | | | (95% CI 0.52-4.01) |
| | | | p for trend = 0.47 |
| | | Worsening | |
| | | of Chair | |
| | | Stand Time | |
| | | (Objective) | |
| Case-based | Tertile 1 (0.74-1.09) | 7/31 (22.6%) | Referent |
| M:L BMD | Tertile 2 (1.09-1.20) | 8/23 (34.8%) | 1.83 |
| Groups | | | (95% CI 0.55-6.09) |
| | Tertile 3 (1.20-1.72) | 8/35 (22.9%) | 1.02 |
| | | | (95% CI 0.32-3.22) |
| | | | p for trend = 0.94 |

TABLE 13
Medial:Lateral BMD Ratios (M:L)

| | | | Odds Ratio |
|---|---|---|---|
| | | Worsening of | |
| | | Total WOMAC | |
| | | Function Score | |
| | | (Subjective) | |
| Case-based | Tertile 1 (0.74-1.09) | 9/31 (29.0%) | Referent |
| M:L BMD | Tertile 2 (1.09-1.21) | 10/25 (40.0%) | 1.63 |
| Groups | | | (95% CI 0.53-4.97) |
| | Tertile 3 (1.21-1.72) | 10/33 (30.3%) | 1.06 |
| | | | (95% CI 0.36-3.11) |
| | | | p for trend = 0.96 |
| | | Worsening of | |
| | | Difficulty | |
| | | Walking | |
| | | (Subjective) | |
| Case-based | Tertile 1 (0.74-1.10) | 4/33 (12.1%) | Referent |
| M:L BMD | Tertile 2 (1.10-1.23) | 5/26 (19.2%) | 1.73 |
| Groups | | | (95% CI 0.41-7.21) |
| | Tertile 3 (1.23-1.72) | 5/30 (16.7%) | 1.45 |
| | | | (95% CI 0.35-6.00) |
| | | | p for trend = 0.62 |

TABLE 14
Proximal Medial:Distal Medial BMD Ratios (PM:DM)

| | | | Adjusted |
|---|---|---|---|
| | | | Odds Ratio |
| | | Worsening | |
| | | of 20-meter | |
| | | Walk Time | |
| | | (Objective) | |
| Case-based | Tertile 1 (1.03-1.32) | 17/38 (44.7%) | Referent |
| M-BMD | Tertile 2 (1.32-1.40) | 18/24 (75.0%) | 3.71 |
| Groups | | | (95% |
| | | | CI 1.20-11.40) |
| (PM:DM) | Tertile 3 (1.40-1.70) | 18/27 (66.7%) | 2.47 |
| | | | (95% CI 0.89-6.88) |
| | | | p for trend = 0.08 |
| | | Worsening | |
| | | of Chair | |
| | | Stand Time | |
| | | (Objective) | |
| Case-based | Tertile 1 (1.03-1.25) | 7/18 (38.9%) | Referent |
| M-BMD | Tertile 2 (1.25-1.40) | 8/44 (18.2%) | 0.35 |
| Groups | | | (95% CI 0.10-1.18) |
| (PM:DM) | Tertile 3 (1.40-1.70) | 8/27 (29.6%) | 0.66 |
| | | | (95% CI 0.19-2.33) |
| | | | p for trend = 0.70 |

TABLE 15
Proximal Medial:Distal Medial BMD Ratios (PM:DM)

| | | | Adjusted |
|---|---|---|---|
| | | | Odds Ratio |
| | | Worsening | |
| | | of 20-meter | |
| | | Walk Time | |
| | | (Objective) | |
| Case-based | Tertile 1 (1.03-1.32) | 17/38 (44.7%) | Referent |
| M-BMD | Tertile 2 (1.32-1.40) | 18/24 (75.0%) | 3.71 |
| Groups | | | (95% |
| (PM:DM) | | | CI 1.20-11.40) |
| | Tertile 3 (1.40-1.70) | 18/27 (66.7%) | 2.47 |
| | | | (95% CI 0.89-6.88) |
| | | | p for trend = 0.08 |
| | | Worsening of | |
| | | Chair Stand | |
| | | Time | |
| | | (Objective) | |
| Case-based | Tertile 1 (1.03-1.25) | 7/18 (38.9%) | Referent |
| M-BMD | Tertile 2 (1.25-1.40) | 8/44 (18.2%) | 0.35 |
| Groups | | | (95% CI 0.10-1.18) |
| (PM:DM) | Tertile 3 (1.40-1.70) | 8/27 (29.6%) | 0.66 |
| | | | (95% CI 0.19-2.33) |
| | | | p for trend = 0.70 |

TABLE 16
Proximal Medial:Distal Medial BMD Ratios (PM:DM)

| | | | Odds Ratio |
|---|---|---|---|
| | | Worsening of | |
| | | Total WOMAC | |
| | | Function Score | |
| | | (Subjective) | |
| Case-based | Tertile 1 (1.03-1.32) | 9/42 (21.4%) | Referent |
| M-BMD | Tertile 2 (1.32-1.40) | 10/23 (43.5%) | 2.82 |
| Groups | | | (95% CI 0.93-8.52) |
| (PM:DM) | Tertile 3 (1.40-1.70) | 10/24 (41.7%) | 2.62 |
| | | | (95% CI 0.88-7.84) |
| | | | p for trend = 0.08 |
| | | Worsening of | |
| | | Difficulty | |
| | | Walking | |
| | | (Subjective) | |
| Case-based | Tertile 1 (1.03-1.35) | 4/49 (8.2%) | Referent |
| M-BMD | Tertile 2 (1.35-1.41) | 5/21 (23.8%) | 3.52 |
| Groups | | | (95% |
| | | | CI 0.84-14.74) |
| (PM:DM) | Tertile 3 (1.41-1.70) | 5/19 (26.3%) | 4.02 |
| | | | (95% |
| | | | CI 0.95-17.04) |
| | | | p for trend = 0.05 |

TABLE 17
Proximal Medial:Total Medial BMD Ratios (PM:TM)

| | | | Odds Ratio |
|---|---|---|---|
| | | Worsening of | |
| | | 20-meter | |
| | | Walk Time | |
| | | (Objective) | |
| Case-based | Tertile 1 (1.01-1.13) | 17/37 (45.9%) | Referent |
| M-BMD | Tertile 2 (1.13-1.16) | 18/25 (72.0%) | 3.03 |
| Groups | | | (95% CI 1.02-8.97) |
| (PM:TM) | Tertile 3 (1.16-1.24) | 18/27 (66.7%) | 2.35 |
| | | | (95% CI 0.84-6.58) |
| | | | p for trend = 0.09 |
| | | Worsening of | |
| | | Chair Stand | |
| | | Time | |
| | | (Objective) | |
| Case-based | Tertile 1 (1.01-1.11) | 7/19 (36.8%) | Referent |
| M-BMD | Tertile 2 (1.11-1.16) | 8/44 (18.2%) | 0.38 |
| Groups | | | (95% CI 0.11-1.27) |
| (PM:TM) | Tertile 3 (1.16-1.24) | 8/26 (30.8%) | 0.76 |
| | | | (95% CI 0.22-2.66) |
| | | | p for trend = 0.72 |

TABLE 18
Pain with Walking:Medial BMD Ratios

| | | Worsening of Pain | |
|---|---|---|---|
| | | during Walking | |
| | | (Subjective) | Odds Ratio |
| Case-Based | Tertile 1 | 5/59 (8.5%) | Referent |
| M-BMD | (1.03-1.39) | | |
| Groups | Tertile 2 | 6/12 (50.0%) | 10.80 |
| (PM:DM) | (1.39-1.42) | | (95% CI 2.52-46.31) |
| | Tertile 3 | 6/18 (33.3%) | 5.40 |
| | (1.42-1.70) | | (95% CI 1.41-20.65) |
| | | | p for trend = 0.006 |
| Case-Based | Tertile 1 | 5/59 (8.5%) | Referent |
| M-BMD | (1.01-1.15) | | |
| Groups | Tertile 2 | 6/13 (46.2%) | 9.26 |
| (PM:TM) | (1.15-1.16) | | (95% CI 2.23-38.45) |
| | Tertile 3 | 6/17 (35.3%) | 5.89 |
| | (1.16-1.24) | | (95% CI 1.52-22.77) |
| | | | p for trend = 0.004 |

No cross-sectional BMD Ratios were associated with any functional assessments. Increased medial BMD ratios were associated with deterioration in walking ability and pain over one year as evidenced by slower walk times, worsening composite WOMAC function scores, worsening reported difficulty walking (WOMAC question) and worsening reported pain while walking (WOMAC question). Increased M:L BMD Ratios were not associated with longitudinal functional decline over one year.

### Example 4

### Increased Medial Tibial Bone Mineral Density (BMD) is Associated with Deterioration in Walking Ability and Pain in Individuals with Knee Osteoarthritis (KOA)

KOA is a major cause of pain and functional limitation in the community, but little is known about factors that predicate clinical progression. However, it is evident that processes in periarticular bone play an important role in KOA progression. Quantitative techniques to measure tibial periarticular BMD show strong cross-sectional relationships with clinical and pathological features of KOA, yet the potential of tibial BMD to predict longitudinal progression has not been tested.

### Methods

The sample was drawn from participants in a trial of vitamin D for KOA who had complete function assessments and WOMAC questionnaire reports at both baseline and 1-year visits. 89 eligible participants had a mean age of 64.2 (±8.7), BMI of 30.3 (±5.3); 67.4% were female. Walking ability was assessed using a timed 20-meter walk test. Pain and difficulty walking were assessed using the 2 pertinent questions from the WOMAC questionnaire. Worsening on the walk test was defined as any increase in walk time from baseline to 1-year, and on the WOMAC questions as any increase in reported severity.

DXA scans of both knees were obtained at baseline using a GE-Lunar scanner. Medial:lateral tibial BMD (M:L BMD) was calculated using a region of interest (ROI) depth of 2 cm, and computed M-BMD ratios in two ways: (1) Ratio of proximal M-BMD to distal M-BMD (PM:DM); and (2) Ratio of proximal M-BMD to total M-BMD (PM:TM). Logistic regression was performed with case-based tertiles of study knee BMD ratios as predictors, and worsening of walk time, walking pain, and walking difficulty as outcomes. Analyses were adjusted for age, sex, BMI, and Kellgren-Lawrence (K/L) grade. These analyses were repeated with K/L grades as predictors.

### Results

Results are shown in Table 19.

TABLE 19

| | | | Adjusted Odds Ratio |
|---|---|---|---|
| | | Worsening of Pain | |
| | | during Walking | |
| | | (Subjective) | |
| Case-based | Tertile 1 | 5/59 (8.5%) | Referent |
| M-BMD | (1.03-1.39) | | |
| Groups | Tertile 2 | 6/12 (50.0%) | 12.87 |
| (PM:DM) | (1.39-1.42) | | (95% CI 2.79-59.48) |
| | Tertile 3 | 6/18 (33.3%) | 7.32 |
| | (1.42-1.70) | | (95% CI 1.48-36.19) |
| | | | p for trend <0.005 |
| | | Worsening of | |
| | | Difficulty Walking | |
| | | (Subjective) | |
| Case-based | Tertile 1 | 4/49 (8.2%) | Referent |
| M-BMD | (1.03-1.35) | | |
| Groups | Tertile 2 | 5/21 (23.8%) | 2.87 |
| (PM:DM) | (1.35-1.41) | | (95% CI 0.63-13.09) |
| | Tertile 3 | 5/19 (26.3%) | 3.31 |
| | (1.41-1.70) | | (95% CI 0.66-16.65) |
| | | | p for trend = 0.12 |
| | | Worsening of | |
| | | 20-meter Walk | |
| | | Time (Objective) | |
| Case-based | Tertile 1 | 17/38 (44.7%) | Referent |
| M-BMD | (1.03-1.32) | | |
| Groups | Tertile 2 | 18/24 (75.0%) | 4.13 |
| (PM:DM) | (1.32-1.40) | | (95% CI 1.30-13.14) |
| | Tertile 3 | 18/27 (66.7%) | 2.74 |
| | (1.40-1.70) | | (95% CI 0.92-8.10) |
| | | | p for trend = 0.06 |

Individuals with higher M-BMD ratios (PM:DM) were significantly more likely to report worse pain at follow-up. Deterioration in walk time and walking difficulty followed the same pattern but were not significant (Table 19). Similar associations were found for PM:TM ratios, but not for M:L BMD ratios. K/L grade was unrelated to any of these measures.

Increased M-BMD ratios are strongly predictive of clinical progression of KOA as gauged by deterioration in walking ability and pain, and are more predictive than radiographs.

### Example 5

### Baseline Vitamin D Status is Predictive of Longitudinal Change in Tibial BMD in Knee Osteoarthritis

With its lack of effective treatment and high prevalence, the public health impact of OA is substantial. Peri-articular bone in OA can be evaluated with the medial:lateral tibial BMD ratio (M:L BMD) obtained from dual x-ray absorptiometry (DXA). Higher M:L BMD is associated with medial OA features on MRI and x-ray.

### Methods:

This is a longitudinal study of participants in a randomized controlled trial (RCT) of vitamin D for symptomatic knee OA. The parent study is ongoing so investigators are still blinded to treatment allocation. Baseline vitamin D levels (ng/mL) were measured. DXA and 1.5 T MRIs of the study knee were obtained at baseline and at 1 year follow-up.

The M:L BMD with a region of interest (ROI) depth of 2 cm were calculated from knee DXAs. The PROXIMAL M:L BMD measuring the proximal lcm of the aforementioned ROI and the DISTAL M:L BMD the distal lcm were also measured. Those in the highest quartile of change in M:L BMD over 1 year were defined as increase in M:L BMD, the middle two as stable M:L BMD, and the lowest as decrease in M:L BMD. The median vitamin D level defined high v. low vitamin D status. To focus on medial disease, those with lateral cartilage damage on MRI were excluded.

Logistic regression was performed with increase in M:L BMD as the outcome and baseline vitamin D as the predictor. Decrease in M:L BMD as the outcome was also investigated. An ordinal logistic regression with increase, stable, and decrease in M:L BMD as the outcome was also performed. All analyses were repeated evaluating the PROXIMAL M:L BMD and the DISTAL M:L BMD.

### Results:

| | | Prevalence of | | | |
|---|---|---|---|---|---|
| | | change in M:L BMD | | | |
| | | | High | | |
| | | Low Vit D | Vit D | | Proportional |
| | | Group | Group | Odds Ratio | Odds Ratio |
| Change in | Increase in M:L | 12/40 | 8/40 | 0.6 | 0.4 |
| M:L BMD | BMD | (30%) | (20%) | (0.2-1.6) | (0.2-0.9) |
| | (0.045-0.158) | | | | |
| | Stable M:L BMD | 23/40 | 17/40 | Referent | |
| | (−0.038-0.041) | (57.5%) | (42.5%) | | |
| | Decrease in M:L | 5/40 | 15/40 | 4.2 | |
| | BMD | (12.5%) | (37.5%) | (1.4-13.1) | |
| | (−0.177-(−0.038)) | | | | |
| Change in | Increase in M:L | 11/40 | 9/40 | 0.8 | 1.0 |
| PROXIMAL | BMD | (27.5%) | (22.5%) | (0.3-2.1) | (0.4-2.3) |
| M:L BMD | (0.081-0.286) | | | | |
| | Stable M:L BMD | 18/40 | 22/40 | Referent | |
| | (−0.006-0.082) | (45.0%) | (55.0%) | | |
| | Decrease in M:L | 11/40 | 9/40 | 0.8 | |
| | BMD | (27.5%) | (22.5%) | (0.3-2.1) | |
| | (−0.122-(−0.006)) | | | | |
| Change in | Increase in M:L | 15/40 | 6/40 | 0.3 | 0.4 |
| DISTAL | BMD | (37.5%) | (15.0%) | (0.1-0.9) | (0.2-0.9) |
| M:L BMD | (0.007-0.080) | | | | |
| | Stable M:L BMD | 18/40 | 22/40 | Referent | |
| | (−0.048-0.007) | (45.0%) | (55.0%) | | |
| | Decrease in M:L | 7/40 | 12/40 | 2.0 | |
| | BMD | (17.5%) | (30.0%) | (0.7-5.8) | |
| | (−0.27-(−0.048)) | | | | |

Participants (N=80) (age 65.7 (±8.6), BMI 30.0 (±5.0), 63.8% female) had a mean vitamin D level of 31.5 ng/mL (+13.3). In those with symptomatic knee OA, a high baseline vitamin D level was associated with a lower odds of increase in M:L BMD and higher odds of decrease in M:L BMD over 1 year. Sufficient change occurred in M:L BMD to detect a difference over 1 year. Results were similar when evaluating the DISTAL M:L BMD but not PROXIMAL M:L BMD. Vitamin D status beneficially influences local changes in bone in knee OA, even in bone somewhat distal from the joint. M:L BMD is useful as a simple inexpensive outcome measure of bone in OA that changes over 1 year.

### Example 6

### Higher Subchondral Bone Volume is Associated with Higher DXA Bone Mineral Density and Knee OA Severity

There is growing evidence that the subchondral bone changes are pathologic in OA. Radiologic imaging allows for visualization of bone in vivo in humans. Apparent bone mineral density (BMD) as measured by Dual X-ray Absoptiometry (DXA) can assess the amount of mineralization within a region of interest (ROI) while MRI is able to measure bone volume fraction (BVF). The relationship of tibial BMD (tBMD) with MRI measured BVF was compared.

### Methods

50 participants of the Osteoarthritis Initiative Bone Ancillary Study who had knee DXAs and knee trabecular bone MRIs obtained at the same visit were included in this study. DXAs were obtained using a customized protocol on GE Lunar Discover Bone Densitometry scanners. Medial proximal tibial BMD (tBMD) including lcm depth of subchondral bone was measured. MRIs were obtained at 3T with 1 mm slice thickness, in-plane spatial resolution of 0.2 mm×0.2 mm, with a 12 cm imaging field-of-view, 512×512 matrix, 72 slice coverage with TE 4.92 msec (fat-water in-phase), TR 20 msec, flip angle 50°, phase right/left, interpolation to 1024×1024, and no partial Fourier. MRIs were analyzed utilizing proprietary software that measured BVF in the medial proximal tibia (tBVF). Results were evaluated for a correlation between medial tBMD and tBVF. Scatter plots of tBMD v. tBVF stratified by JSN and ran ANOVAs of tBMD and tBVF by JSN were then created.

### Results

The mean age was 67.2 (9.5), BMI 28.1 (4.1), and 50% were male. 31 had JSN of 0, 14 with JSN of 1, and 5 with JSN of 2. The correlation between the medial tBMD and tBVF was r=0.64, p<0.0001. The tBMD by JSN were 1.17 g/cm², 1.30, and 1.60 for JSN 0, 1, and 2 respectively, p=0.0003. The tBVF by JSN were 0.15, 0.22, and 0.29 for JSN 0, 1, and 2 respectively, p=0.0042. FIG. 5 summarizes the results of the present example.

In those with symptomatic knee OA, a high baseline vitamin D level was associated with a lower odds of increase in M:L BMD and higher odds of decrease in M:L BMD over 1 year. Sufficient change occurred in M:L BMD to detect a difference over 1 year. Results were similar when evaluating the DISTAL M:L BMD but not PROXIMAL M:L BMD. Vitamin D status seems to beneficially influence local changes in bone in knee OA, even in bone somewhat distal from the joint.

Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of determining a risk of osteoarthritis in a subject, comprising:
a) determining one or more ratios of bone mineral density in a region of a joint bone of said subject selected from the group consisting of medial bone mineral density: lateral bone mineral density, medial proximal bone mineral density: medial bone mineral density, and proximal medial bone mineral density: distal medial bone mineral density; and
b) identifying subjects at risk of developing osteoarthritis when said ratio of bone mineral density is increased relative to the level in subjects not known to develop osteoarthritis.

2. The method of claim 1, wherein said joint is a knee joint.

3. The method of claim 1, wherein said bone mineral density is determined using dual X-ray absorptiometry (DXA).

4. The method of claim 1, wherein a medial proximal bone mineral density: medial bone mineral density greater than 1.32 is indicative of subjects at risk of developing osteoarthritis.

5. A method of monitoring progression of osteoarthritis in a subject diagnosed with osteoarthritis, comprising:
a) determining one or more initial ratios of bone mineral density at an initial time point in a region of a joint bone of said subject selected from the group consisting of medial bone mineral density: lateral bone mineral density, medial proximal bone mineral density: medial bone mineral density, and proximal medial bone mineral density: distal medial bone mineral density;
b) determining a second one or more initial ratios of bone mineral density at a later time point in a region of a joint bone of said subject selected from the group consisting of medial bone mineral density: lateral bone mineral density, medial proximal bone mineral density: medial bone mineral density, and proximal medial bone mineral density: distal medial bone mineral density; and
c) identifying subjects as having a progression of osteoarthritis when said ratio of bone mineral density is increased at said later time point relative to said initial time point.

6. The method of claim 5, wherein a medial proximal bone mineral density: medial bone mineral density greater than 1.32 is indicative of subjects at risk of having progression of osteoarthritis.

7. The method of claim 5, wherein said joint is a knee joint.

8. The method of claim 5, wherein said bone mineral density is determined using dual X-ray absorptiometry (DXA).

9. The method of claim 5, wherein said later time point is approximately one year after said initial time point.

10. The method of claim 5, further comprising the step of determining a further one or more initial ratios of bone mineral density at later time points in a region of a joint bone of said subject selected from the group consisting of medial bone mineral density: lateral bone mineral density, medial proximal bone mineral density: medial bone mineral density, and proximal medial bone mineral density: distal medial bone mineral density.

11. The method of claim 10, wherein said later time points are spaced approximately one year apart.

12. The method of claim 5, further comprising the step of administering a test compound to said subject.

13. A system, comprising:
a) an imaging device; and
b) computer hardware and software configured to calculate bone mineral density in a region of a joint bone of a subject selected from the group consisting of medial bone mineral density: lateral bone mineral density, medial proximal bone mineral density: medial bone mineral density, and proximal medial bone mineral density: distal medial bone mineral density; and
c) a used interface configured to display said bone mineral density ratios.

14. The system of claim 13, wherein said imaging device is a DXA device.

15. The system of claim 13, wherein said imaging device determines said region of a joint bone.

16. The system of claim 13, wherein said computer hardware maintains a database of bone mineral density ratio.

17. The system of claim 16, wherein said bone mineral density ratios in said database are tagged with subject identification tags and time stamp tags.
